(19) United States
(12) Patent Application Publication
(10) Pub. No.: US 2011/0196195 A1
(43) Pub. Date: Aug. 11, 2011

(54) IMPLANTABLE SUBCUTANEOUS ACCESS PORT

(75) Inventors: Joseph S. Raven, Goleta, CA (US); Nikhil S. Trilokekar, Goleta, CA (US); Kaustubh S. Chitre, Goleta, CA (US)

(73) Assignee: ALLERGAN, INC., Irvine, CA (US)

(21) Appl. No.: 12/948,703

(22) Filed: Nov. 17, 2010

Related U.S. Application Data
(60) Provisional application No. 61/301,910, filed on Feb. 5, 2010.
(60) Provisional application No. 61/409,440, filed on Nov. 2, 2010.
(60) Provisional application No. 61/409,449, filed on Nov. 2, 2010.

(51) Int. Cl. A61F 2/04

(52) U.S. Cl. 600/37

(57) ABSTRACT

The present invention generally provides a gastric banding system, including an implantable access port having a substantially ellipsoid shape. The access port is pliable and smooth, to increase the comfort of the access port to the patient, and to increase the aesthetic effect of the access port. The ellipsoid shape provides a large needle penetrable surface area for the access port, and enhances the ability of a physician to detect the access port through tactile means. A mesh layer may be incorporated in the housing of the access port, to increase durability of the housing, and to promote the self-sealing properties of the housing.

### RELATED APPLICATIONS

This application claims priority to and the benefit of U.S. Provisional Patent Application No. 61/301,910, entitled “TISSUE EXPANDER” filed on Feb. 5, 2010, the entire disclosure of which is incorporated herein by reference.

### FIELD

The present invention generally relates to medical systems and apparatus and uses thereof for treating obesity and/or obesity-related diseases, and more specifically, relates to an implantable subcutaneous access port.

### BACKGROUND

Adjustable gastric banding apparatus have provided an effective and substantially less invasive alternative to gastric bypass surgery and other conventional surgical weight loss procedures. Despite the positive outcomes of invasive weight loss procedures, such as gastric bypass surgery, it has been recognized that sustained weight loss can be achieved through a laparoscopically-placed gastric band, for example, the LAP-BAND® (Allergan, Inc., Irvine, Calif.) gastric band or the LAP-BAND AP® (Allergan, Inc., Irvine, Calif.) gastric band. Generally, gastric bands are placed about the fundus, or cardia, or esophageal junction, of a patient's upper stomach forming a stoma that restricts food's passage into a lower portion of the stomach. When the stoma is of an appropriate size that is restricted by a gastric band, the food held in the upper portion of the stomach may provide a feeling of satiety or fullness that discourages overeating. Unlike gastric bypass procedures, gastric band apparatus are reversible and require no permanent modification to the gastrointestinal tract. An example of a gastric banding system is disclosed in Roslin, et al., U.S. Patent Pub. No. 2006/0235448, the entire disclosure of which is incorporated herein by this specific reference.

Existing gastric bands periodically require adjustments to maintain an effective constriction about the stomach, to account for changes in the stomach tissue, reduction of fat or other factors causing movement and/or size change of the stomach. Some attempts have been made to allow for such adjustment of gastric bands. For example, hydraulic gastric bands utilize a fluid such as saline to fill an inflatable portion of the gastric band using a subcutaneous injection access port. Adjustments to the amount of inflation may be made by injecting or extracting the fluid through the patient's skin into or out of the injection access port, which then directs the fluid into or out of the inflatable portion of the gastric band.

Current access ports are typically configured to include a flat-surfaced septum, a rigid housing, and a substantially flat base. The housing forms a rim around the septum to expose a circular surface area of the septum.

U.S. Pat. No. 6,997,914 issued to Smith and Fowler describes an implantable access port comprising a self-sealing, penetrable septum secured onto the base of the access port by a retainer ring, wherein the base and the retainer ring are preferably composed of a biocompatible material, such as electro-polished stainless steel or hard material such as titanium, and a dome-shaped reservoir inside the port.

U.S. Pat. No. 5,041,098 issued to Loiterman et al. describes an implantable access port comprising a self-sealing, penetrable septum affixed to a housing, wherein the housing is preferably formed of hard materials such as titanium or surgical grade steel and includes a lip which lines the peripheral portion of the septum.

U.S. Pat. No. 4,190,040 issued to Schulte describes a subcutaneous injection housing that has a generally dome-shaped sealant chamber which can reseal needle punctures in the housing and has a generally flat base.

U.S. Pat. No. 4,543,088 issued to Bootman and Yamamoto describes a resealable puncture housing for surgical implantation having a generally dome-shaped wall, which comprises an upper wall and a side wall, wherein the outer surface of the upper wall has a substantially flat surface and the side wall is generally straight and vertical.

The access ports currently on the market suffer from a known series of drawbacks, including patient discomfort due to the shape or rigidity of the subcutaneously implanted access port, and patients' aesthetic concerns about the appearance of the access port. A rigid access port may press against, and damage, portions of the patient's body during the natural body motion of the patient. In addition, the rigid construction of access ports currently on the market may serve as an irritant beneath the patient's skin. In addition, the rim, housing, and septum configuration of many access ports on the market may produce an aesthetic concern for the patient. As the patient loses weight over time, the access port will gradually become more visible through the patient's skin. The rim of the septum may exhibit a mechanical or ring-like shape beneath the patient's skin, which may embarrass the patient.

In addition, access ports currently on the market may be difficult to locate subcutaneously by palpation or other means, and may be difficult to penetrate with syringe needles. Physicians may not be able to easily detect the flat upper surface of an access port currently on the market, located beneath many layers of fat. In addition, once the physician detects the access port, the physician must attempt to pass a syringe through the layers of fat, and into a septum, which may comprise a relatively small proportion of the total outer surface area of the housing. The physician may miss the septum entirely and may contact internal organs of the patient, causing great pain. In addition, the physician may contact the tubing leading from the access port, causing the tubing to rupture, and requiring surgery to mend the tube, or to replace the entire gastric banding system.

Accordingly, it is desirable to develop a gastric banding system, and an implantable access port designed to remedy the deficiencies of access ports currently on the market.

### SUMMARY

Generally described herein is a gastric banding system, including an implantable access port having a substantially ellipsoid shape. The access port is pliable and smooth, to increase the comfort of the access port to the patient, and to increase the aesthetic effect of the access port. The ellipsoid shape provides a large needle penetrable surface area for the access port, and enhances the ability of a physician to detect the access port through tactile means.

The access port includes a housing comprising an ellipsoid-shaped shell defining an internal fluid reservoir. The shell has an anterior side and a posterior side, the anterior side being the side of the housing facing the patient's outer skin layer, and the posterior side being the opposite side of the housing, facing away from the patient's outer skin layer. The outer surface of the anterior side of the housing may be made entirely from a needle penetrable and self-sealing material. A portion of the posterior side of the housing may be made from a needle penetrable and self-sealing material, and a portion of the posterior side of the housing may be made from a needle-resistant material. The needle penetrable and self-sealing material of the anterior side and/or posterior side preferably comprises a pliant material, to provide a degree of compliance for the housing.

A mesh layer may be included in a portion of the anterior side of the housing and/or in a portion of the posterior side of the housing. The mesh layer of the anterior side of the housing may be contiguous with the mesh layer of the posterior side of the housing. The mesh layer serves to enhance the self-sealing properties of the housing, by constraining the materials of the housing, to prevent fluid from leaking from the reservoir.

A layer of needle resistant material may cover a portion of the posterior side of the housing, or the entire posterior side of the housing. The needle resistant material may also cover a portion of the anterior side of the housing. The needle resistant material may be positioned adjacent to the fluid reservoir, to reduce the possibility of a syringe needle passing through the housing, once it enters the reservoir.

An anchor member is fixed to a portion of the housing, preferably the posterior side of the housing. The anchor member may comprise a mesh member made of a mesh material, being biocompatible. The mesh material may be capable of integrating with the local body tissue of the patient. The mesh material may be sutured or tacked to the patient's body. The anchor member may have a skirt-like, sheet-like, or disk-like shape, and may extend out from the housing at a diameter being larger than the diameter of the housing. In one embodiment, the mesh material of the anchor member may be contiguous with the mesh layer of the posterior side of the housing and/or the mesh layer of the anterior side of the housing.

A connecting tube extends from a conduit in the housing. The connecting tube transfers fluid between the gastric band and the fluid reservoir. The connecting tube may extend from the posterior side of the housing, and, in one embodiment, may pass through a portion of the needle resistant material, and/or the anchor member. The connecting tube may extend from the posterior side of the housing at an angle, preferably an acute angle. The angle reduces erosion of local tissue and organs caused by the connecting tube. In addition, if the tube passes beneath the needle resistant material and/or the anchor member, then the needle resistant material and/or anchor member may shield the connecting tube from puncture by an incoming syringe needle. In one embodiment, the anchor member may be integrated with a ring, which offers further protection for the connecting tube.

The implantable access port is designed to overcome known deficiencies of access ports currently on the market, including pain associated with the access port, aesthetic concerns associated with the access port, difficulty in locating the access port, difficulty in locating a penetrable surface of the access port, and difficulty penetrating the access port with a syringe.

The access port is preferably made of soft, or pliant, materials that allow the housing of the access port to flex or deform when a force is exerted against the housing. The pliant nature of the access port may reduce the pain associated with a rigid device placed within a patient's body. In addition, the surface of the access port housing has a substantially curved profile, which reduces the number of sharp angles or edges of the access port. Fewer angles or edges in the patient's body may reduce damage caused by the access port.

The access port also has a smooth, curved outer shell, which produces a more organic appearance than the rim, housing, and septum combination, of known gastric bands. The organic appearance may increase the aesthetics of the access port, if the access port is visible through the skin of the patient. The outline of the access port may appear invisible, or less visible than access ports currently on the market.

The access port also reduces the difficulty in locating the access port through tactile means. The access port has an ellipsoid shape, which provides a distinct volume and structure, which may be easily identified and detected by a physician.

The access port also increases the total penetrable surface area of the housing beyond the septum size of access ports currently on the market. The outer surface of the housing effectively comprises the septum of the housing, and is needle penetrable from various incident angles. Currently, access ports on the market commonly employ a substantially flat-surfaced septum as the facet of the port available for percutaneous needle insertion. A rigid housing forms an impenetrable rim around the septum. In these access ports, the proportion of the port accessible for percutaneous needle insertion is limited to a central, circular region on top of the housing. The total needle penetrable surface of the present invention's access port may be between two times and three times larger than access ports currently on the market (e.g., access ports currently on the market may have a septum size of approximately 0.17 square inches, the present invention contemplates a septum size of approximately 0.45 square inches).

In addition, access ports currently on the market may be available for percutaneous top-down entry of a syringe needle, only if the penetrable septum is facing, or is parallel to the surface of the skin. As a result, if such an access port inverts or otherwise changes its usual orientation such that the exposed septum surface is no longer facing or is parallel to the surface of the skin, a needle cannot enter the port to deliver fluids. The access port is penetrable from multiple angles, including angles along the sides of the housing, which allow the access port to remain functional even if it varies its orientation within the patient's body.

The access port of the present invention, in at least one embodiment, increases the protection offered by the access port for the connecting tube. The connecting tube may pass beneath the housing, the needle resistant material, and/or the anchor member, to allow these structures to further protect the tube from puncture by incident syringe needles. The connecting tube may also pass underneath a ring of the anchor member, which further protects the tube from puncture by incident syringe needles.

### BRIEF DESCRIPTION OF THE DRAWINGS

The exemplary embodiments of the present invention will be described in conjunction with the accompanying drawing FIGS. in which like numerals denote like elements and:

FIG. 1 illustrates a perspective view of a gastric banding system according to an embodiment of the present invention;

FIG. 2 illustrates a cross-sectional view of an access port according to an embodiment of the present invention;

FIG. 2A illustrates a close-up cross-sectional view of a portion of the anterior side of the access port housing according to an embodiment of the present invention;

FIG. 3 illustrates a perspective view of an access port displaying a x-axis, a y-axis, and a z-axis of the access port housing according to an embodiment of the present invention;

FIG. 4 illustrates a cross-sectional view of an access port according to an embodiment of the present invention;

FIG. 5 illustrates a cross-sectional view of an access port according to an embodiment of the present invention;

FIG. 6 illustrates an exploded perspective view of an access port according to an embodiment of the present invention;

FIG. 7 illustrates a cross-sectional view of an access port according to an embodiment of the present invention; and

FIG. 8 illustrates a side view of an access port according to an embodiment of the present invention.

### DETAILED DESCRIPTION

The present invention generally provides a gastric banding system, including an implantable access port having a substantially ellipsoid shape. The access port is pliable and smooth, to increase the comfort of the access port to the patient, and to increase the aesthetic effect of the access port. The ellipsoid shape provides a large needle penetrable surface area for the access port, and enhances the ability of a physician to detect the access port through tactile means.

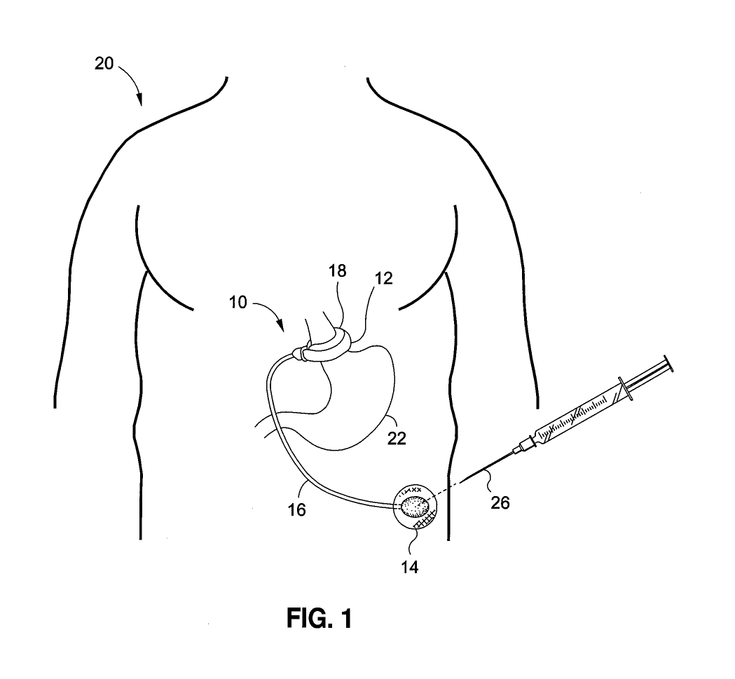

FIG. 1 illustrates a gastric banding system 10 contemplated for use in the present invention, comprising a gastric band 12, an access port 14, and a connecting tube 16. The gastric band 12 may include a lumen, an inflatable portion, or inflatable bladder 18, which is capable of being filled with fluid. The connecting tube 16 couples the bladder 18 to the access port 14 and supplies fluid to the inflatable bladder 18.

The gastric band 12 forms a loop around a portion of a patient's, or individual's 20 stomach 22. The portion of the stomach 22 receiving the gastric band 12 may comprise the cardia, the fundus, or the esophageal junction of an individual's 20 stomach 22. The loop constricts a portion of the individual's 20 stomach 22 to form a stoma, which restricts the flow of food entering the lower portion of the stomach 22 when the individual 20 eats. The restricted flow of food promotes a more rapid production of satiety signals during times of food consumption, than are normally produced without the restricted flow of food. The increased satiety signals hopefully cause the individual 20 to feel full more quickly, and cause the individual 20 to reduce food intake. The reduced food intake hopefully causes the individual 20 to lose weight over time.

It is preferable if the degree of constriction applied by the gastric band 12 to the stomach 22 is variable. A variable degree of constriction is preferred because the biological characteristics of an individual's 20 stomach 22 may vary over time. For example, the individual's 20 stomach 22 may increase or decrease in size, requiring an appropriately larger or smaller degree of constriction. In addition, the degree of constriction may need to be varied if the individual 20 is not losing weight in response to the gastric band treatment, or if the weight loss is not at a desired level. If the individual 20 is not responding appropriately to the gastric band therapy, the gastric band 12 may need to be adjusted, to increase the degree of constriction, and to further decrease the flow of food to the lower portion of the stomach. In addition, it is preferable if the size of the gastric band 12 itself is variable, to accommodate the unique biological characteristics of different patients, for example, patients having a smaller or larger sized stomach.

To accommodate a varying degree of constriction, or varying gastric band 12 size, the gastric band 12 may comprise an adjustable gastric band 12, or comprise a gastric band 12 having an adjustable diameter. The gastric band 12 may be adjusted by adjusting the amount of fluid in the bladder 18. The bladder 18 may extend around the portion of the stomach 22 to be constricted, and may have a variable size, functioning as an inflatable cuff placed around the stomach 22. An increased bladder 18 size may increase the degree of constriction applied to the stomach 22, and a decreased bladder 18 size may decrease the degree of constriction. Accordingly, an increased amount of fluid in the bladder 18 may increase the degree of constriction, and a decreased amount of fluid in the bladder 18 may decrease the degree of constriction.

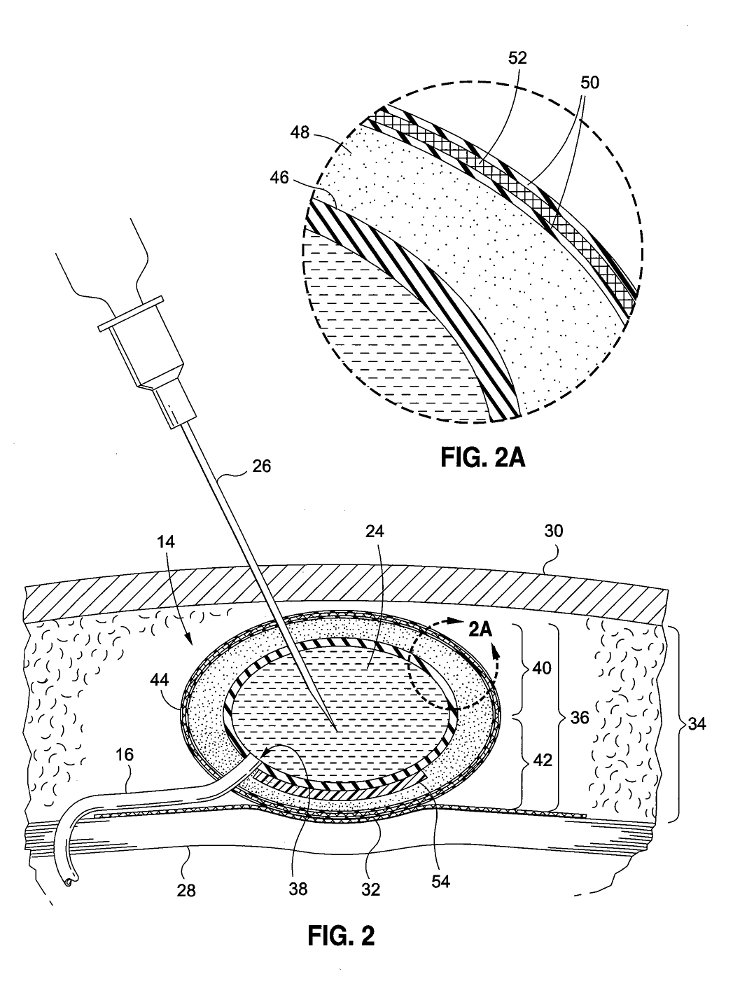

The amount of fluid in the bladder 18 may be controlled via the access port 14. The access port 14 is preferably positioned subcutaneously within the individual's 20 body and is preferably secured to a firm layer of tissue, for example, the individual's 20 muscle wall. The access port 14 may include an internal fluid reservoir 24, as shown in FIG. 2, in fluid communication with the bladder 18 of the gastric band 12, through the connecting tube 16. If more fluid is injected into the reservoir 24, then the bladder 18 increases in size, and the degree of constriction is increased. If fluid is removed from the reservoir 24, then the bladder 18 decreases in size, and the degree of constriction decreases. A physician accesses the access port 14 through the skin of the patient, to vary the amount of fluid in the fluid reservoir 24. The physician inserts a syringe 26 through the skin of the patient, to penetrate the access port 14, and add or remove fluid from the fluid reservoir 24. The access port 14 is therefore preferably positioned near the surface of the individual's 20 skin, to allow a physician to more easily access the access port 14 with the syringe 26.

FIG. 2 illustrates an embodiment of the access port 14 shown in FIG. 1. The access port 14 is shown coupled to the muscle wall 28 of the individual, beneath the individual's skin 30. An anchor member 32 connects the access port 14 to the muscle wall 28. A fat layer 34 surrounds the access port 14.

The access port 14 includes a housing 36 that has a substantially ellipsoidal shape, and defines a reservoir 24 within the interior of the housing 36. A fluid conduit 38 is positioned within the housing 36 and connects the fluid reservoir 24 to the connecting tube 16. The connecting tube 16 is coupled to the gastric band 12, as shown in FIG. 1.

The housing 36 defines a shell around the fluid reservoir 24. The shell is formed by the combination of two half-shells, one half-shell being defined by an anterior side 40 of the housing 36 and other half-shell being defined by a posterior side 42 of the housing 36. The anterior side 40 includes the portion of the access port 14 positioned towards the individual's skin 30, and above the midline 44 of the access port 14, which represents a maximal width of the access port 14, formed due to the ellipsoidal shape of the access port 14. The anterior side 40 is the top-half of the housing 36, and has a substantially dome-like, or half-dome shape. Once the access port 14 is implanted beneath the skin 30 of a patient, the anterior side 40 of the housing 36 comprises a penetrable surface that faces, and is positioned directly beneath, the patch of skin 30 through which a syringe 26 needle can be inserted, to inject fluid into the access port 14. The posterior side 42 includes the portion of the access port 14 positioned towards the individual's muscle wall 28, and below the midline 44 of the access port 14. The posterior side 42 is the bottom-half of the housing 36, and has a substantially dome-like or half-dome shape.

The anterior side 40 of the housing 36 is made from at least one material being needle penetrable and self-sealing. The anterior side 40 of the housing 36 may include a single layer of material, or multiple layers of materials placed adjacent to each other. The single layer of material, or multiple layers of material are preferably resilient, but firm enough to enable detection of the access port beneath the skin by palpation. A layer of material may include silicones of scale (A) durometer 65+/−35 and scale (D) durometer 50+/−20, with a thickness greater than 0.080 inches. The layer or layers of material are preferably needle penetrable and self-sealing because the anterior side 40 must sustain repeated, periodic insertion and withdrawal of needles without leakage of fluid from inside the access port 14. The layer or layers are preferably made of a biocompatible material, that is nontoxic and not harmful to mammals. Silicone elastomer, such as a dimethyl or dimethyl-diphenyl silicone elastomer, may be used in a single layer or multiple layers of the anterior side 40.

Referring to FIG. 2A, the anterior side 40 of the housing 36 may comprise multiple layers of material, including an interior layer 46, an intermediate silicone gel layer 48, an exterior layer 50, and a mesh layer 52. The interior layer 46 defines an outer boundary of the fluid reservoir 24, and may extend around and encompass the fluid reservoir 24. The interior layer 46 of the anterior side 40 may have a half-dome shape, similar to the shape of the anterior side 40 of the housing 36. The interior layer 46 of the anterior side 40 may extend entirely around the anterior side 40 of the fluid reservoir 24. The interior layer 46 is preferably made from an elastomeric material. The elastomeric material is needle penetrable and self-sealing, to withstand repeated and periodic insertion and withdrawal of needles, without causing fluid to leak from the fluid reservoir 24.

The silicone gel layer 48 extends over, adjacent to, and around the interior layer 46, encircling the interior layer 46. The silicone gel layer 48 comprises a soft and pliable layer of silicone gel extending around the interior layer 46. The silicone gel layer 48 may aid the interior layer 46 to prevent fluid from leaving the reservoir 24, by providing a barrier layer to the fluid. In addition, the soft and malleable nature of the gel layer 48 also increases the overall softness and pliability of the housing 36, and the access port 14.

The exterior layer 50 may comprise two layers of elastomeric material placed adjacent to, or on both sides of, a mesh layer 52. The exterior layer 50 defines the outer boundary, or outer surface of the housing 36. The anterior side 40 portion of the exterior layer 50 may have a shape that defines the half-dome shape of the anterior side 40. The elastomeric materials comprising the two layers of the exterior layer 50 may have similar properties as the material comprising the interior layer 46, namely, the elastomeric materials of the exterior layer 50 may be needle penetrable and self sealing.

The embedded mesh layer 52 may be integrated, or embedded, within the exterior layer 50. The embedded mesh layer 52 enhances the resealability of the exterior layer 50, and adds a degree of structure to the housing 36, allowing the housing 36 to retain an ellipsoid shape after manipulation by a physician, insertion of needles, and contact with other elements of the surrounding environment (e.g., other parts of the interior of the patient's body, or forces exerted from outside the patient's body). The mesh layer 52 may act in combination with the gel layer 48 to enhance the resealability of the housing 36 shell. For example, after a void is created in the shell by a needle used to introduce fluid into the reservoir 24, the gel layer 48 prevents the fluid from having a direct path to the exterior of the housing 36. The mesh layer 52 enhances this property of the gel layer 48 by physically constraining the gel from expansion under pressure exerted by the fluid within the reservoir 24. The material used in the mesh layer 52 may comprise polyethylene terephthalate, polypropylene, polyamide, polyethylene, and combinations thereof. The material used in the mesh layer 52 may also comprise any other material capable of producing an equivalent result.

Referring back to FIG. 2, the posterior side 42 of the housing 36 may also have a similar layered composition as the anterior side 40. For example, the posterior side 42 may include an interior layer 46 being made from an elastomeric material. The interior layer 46 may extend entirely around the posterior side 42 of the fluid reservoir 24, notwithstanding the position of the conduit 38 extending through the interior layer 46. The elastomeric material of the posterior side's 42 interior layer 46 may be needle penetrable and self-sealing. In addition, the posterior side 42 may include a silicone gel layer 48 positioned adjacent to the interior layer 46. The posterior side 42 may also include an exterior layer 50 with an embedded mesh 52. The exterior layer 50 may have a half-dome shape that defines the half-dome shape of the posterior side 42. The interior layer 46, gel layer 48, exterior layer 50, and mesh 52 of the posterior side 42 may have a similar configuration and composition as the interior layer 46, gel layer 48, exterior layer 50, and mesh 52 of the anterior side 40. The elastomeric material of the posterior side's 42 exterior layer 50 may be needle penetrable and self-sealing.

The posterior side 42 may also include one or more layers of needle-resistant material 54 extending around a portion of the fluid reservoir 24. The single layer or multiple layers of needle-resistant material 54 may be positioned outside the fluid reservoir 24 adjacent to the interior layer 46 (as shown in FIG. 2). The needle-resistant material 54 may be bonded to the interior layer 46, within the shell of the housing 36. The needle-resistant material 54 may have a curved shape, to contour to the shape of the interior layer 46, or the fluid reservoir 24. In one embodiment, the needle-resistant material 54 may be bonded to the exterior layer 50. In one embodiment, the entirety of the posterior side 42, or a portion of the posterior side 42 may comprise only a single layer or multiple layers of needle-resistant material.

The layer of needle-resistant material 54 may comprise any biocompatible reinforced fabric, or textile. For example, the needle-resistant material 54 may comprise any suitable biocompatible polymer available in the art, such as KEVLAR®, with sufficient thickness to resist puncturing. Other materials may include specialty material, specialty woven material, and reinforced fabrics. Examples of other needle-resistant materials that may be used include polyethylene, polypropylene, polyimide, thermoplastic polyurethanes, higher durometer silicone, acrylonitrile butadiene styrene, and the like. It is contemplated that various other materials may be utilized that produce an equivalent result.

In one embodiment, the anterior side 40 and the posterior side 42 may be constructed of the same layers of material, although the posterior side 42 additionally contains a layer of needle-resistant material 54. In one embodiment, the anterior side 40 may comprise a layer or layers of material being different than a layer or layers of material of the posterior side 42. For example, the posterior side 42 may comprise only a layer of needle-resistant material 54 and the anterior side 40 may comprise the exterior layer 50 containing mesh 52, the intermediate layer of silicone gel 48, and the interior layer 46 of elastomeric material. In addition, in one embodiment, the mesh material used in the mesh layer 52 of the anterior side 40 and posterior side 42 may be different. In addition, in one embodiment, the mesh material used in the mesh layer 52 of the anterior side 40 and the posterior side 42 may be made of the same material, and contiguous. In addition, in one embodiment, the shell of the housing 36 may comprise a dual-lumen, gel-filled silicone, without an integrated mesh layer 52. In addition, in one embodiment, the entire shell of the housing 36 may comprise a singular silicone layer. Various combinations of materials and layers may be used in the anterior side 40 and the posterior side 42 to produce a desired result.

The anterior side 40 and the posterior side 42 may be manufactured separately, then sealed together to form the shell of the housing 36. The anterior side 40 and the posterior side 42 may be sealed together through ultrasonic welding, or other equivalent techniques. In one embodiment, the entire housing 36, including the anterior side 40 and the posterior side 42, may be constructed as a whole, and may not require separate sides 40, 42 to be fused together. The housing 36 is preferably formed as a whole when the anterior side 40 and the posterior side 42 are made from similar material layers, notwithstanding the additional needle-resistant material 54 present in the posterior side 42. To manufacture the housing 36 as a whole, a dipping method may be used. The dipping method may be used to form an embodiment of the housing 36 including an exterior layer 50 having an embedded mesh 52, an intermediate silicone gel layer 48, an interior layer 46, and a layer of needle resistant material 54, as shown in FIG. 2.

The dipping method generally comprises making the exterior layer 50 of the housing 36 by dipping two or more layers of silicone-based elastomer over a mandrel in the desired shape of the housing 36, for example, a substantially ellipsoid shape. A pre-fabricated mesh is placed over the formed elastomer layers, followed by two or more dips of the silicone-based elastomer. The mesh is then embedded between layers of elastomer, as shown in FIG. 2A. The entire assembly of the exterior layer 50 is then heated in an oven at a temperature and for a period of time suitable to cure the silicone. Post-curing, the exterior layer 50 of the housing is removed from the mandrel. The interior layer 46 is formed in a similar manner, by dipping layers of silicone-based elastomer over a mandrel.

The interior layer 46 is placed inside the exterior layer 50, and the exterior layer 50 and the interior layer 46 are vulcanized. A compartment is formed between the exterior layer 50 and the interior layer 46. The assembly of the exterior layer 50 and the interior layer 46 is then mounted back on a mandrel. The size the mandrel may be the same as the one used for the fabrication of the interior layer 46 or slightly larger, as a slightly larger mandrel would result in a laterally stressed inner shell with potentially enhanced sealing properties. The compartment formed between the exterior layer 50 and the interior layer 46 is then filled with silicone gel. This may be accomplished using any suitable method known to those skilled in the art. The silicone gel between the exterior layer 50 and the interior layer 46 forms the intermediate gel layer 48. After the compartment is filled with gel, the assembly of the exterior layer 50, the interior layer 46, and the gel layer 48 is exposed to heat in an oven for a suitable period of time. Before sealing the housing 36 with a patch, a needle-resistant material 54 is inserted and bonded to the interior layer 46 and/or the exterior layer 50.

The housing 36 encloses a space, or cavity, that serves as the fluid reservoir 24. The interior layer 46 extends around and encompasses the fluid reservoir 24. The fluid reservoir 24 may have the same shape as the housing 36 (e.g., an ellipsoid shape). The reservoir 24 may also have varied equivalent shapes capable of retaining a measure of fluid. The pressure inside the reservoir 24 may range from 0 mmHg to 800 mmHg, although it is contemplated this pressure range may be varied as desired.

In the embodiment shown in FIG. 2, the anchor member 32 comprises a mesh member that is directly joined to the posterior side 42 of the housing 36. The anchor member 32 attaches the access port 14 to body tissue. The anchor member 32 may be secured to the posterior side 42 of the housing 36 by ultrasonic welding or other suitable means. The mesh anchor member 32 preferably has a substantially symmetrical shape, such as a square or a circle. Only a portion of the mesh anchor member 32, preferably a central portion, is joined to the posterior side 42. The portion of the mesh anchor member 32 that is not joined to the posterior side is available for attachment to subcutaneous tissue (e.g., the muscle wall 28) via sutures or surgical tacks, or other equivalent means. The mesh anchor member 32 may also connect to subcutaneous tissue by merely placing the anchor member 32 on the desired fixing point of the tissue, and allowing local tissue to integrate with the mesh, fixing the anchor member 32 in place. The mesh may also be flexible, to allow the physician to manipulate the mesh during implantation. In addition, a flexible mesh may allow the anchor member 32 to be contoured to fit along varied shapes of the patient's body to which it is fixed. The mesh material is preferably biocompatable to allow the anchor member 32 to integrate with the tissue of the patient's body, and strengthen the body's bond to the access port 14, and increase the body's biological acceptance of the access port 14 (e.g., reducing the chance of infection and other signs of biological rejection of the port). The mesh may be made from a material comprising polyethylene terephthalate, polypropylene, polyamide, polyethylene, and combinations thereof. The mesh material of the anchor member 32 may be the same material used as the embedded mesh 52 of the anterior side 40, or the posterior side 42, or may comprise a different mesh material.

The conduit 38 comprises a channel, or passageway through the housing 36, that allows fluid to enter or exit the fluid reservoir 24 from or to the exterior of the housing 36, respectively. The conduit 38 may be positioned near the posterior side 42 of the housing 36, as shown in FIG. 2, or may be positioned in varied locations along the fluid reservoir 24 that may provide an equivalent result (e.g., near a lower portion of the anterior side 40 of the housing 36). The conduit 38 allows fluid within the reservoir 24 to pass through the layer, or layers of material surrounding the reservoir 24, and to pass to the gastric band 12, as shown in FIG. 1. The conduit 38 is preferably positioned near the posterior side 42 of the housing 36 to allow the connecting tube 16 to extend from the conduit 38 in a direction away from a syringe 26 inserted by a physician.

The connecting tube 16 connects to the conduit 38, and fluidly couples the reservoir 24 to the gastric band 12 (as shown in FIG. 1). As shown in FIG. 2, the connecting tube 16 may extend from the posterior side 42 of the housing 36. The connecting tube 16 extends in a direction away from the anterior side 40 of the housing 36, to reduce the possibility that the connecting tube 16 is punctured by an incoming syringe 26. The connecting tube 16 may extend along the surface of the anchor member 32 and then pass through a portion of the patient's body (e.g., the muscle wall 28) to connect to the gastric band 12. The connecting tube 16 may be made from silicone elastomer, or the equivalent. The connecting tube 16 may be of any length suitable to connect the access port 14 to the gastric band 12, depending on the desired placement of the access port 14 and the gastric band 12. For example, the connecting tube 16 may have a length of five inches, if the access port 14 is placed along the abdominal muscle sheath of the patient.

In operation, the access port 14 receives a syringe 26 inserted through the skin 30 of the patient. The syringe 26 penetrates the layer, or multiple layers, of the anterior side 40 of the housing 36 until the syringe 26 enters the fluid reservoir 24. The syringe 26 may also enter the housing 36 through the posterior side 42 of the housing 36, if the syringe 26 does not contact the needle resistant material 54. The syringe 26 deposits or removes fluid from the fluid reservoir 24. When the syringe 26 is withdrawn from the housing 36, the composition of the anterior side 40 of the housing 36, or possibly the posterior side 42 of the housing 36, prevents fluid from exiting the reservoir 24.

The construction of the access port 14 provides benefits that aid both the physician and patient in the use and comfort of the access port 14. For example, the layer of needle-resistant material 54 is positioned along the posterior side 42 of the housing 36 to prevent the syringe 26 needle from penetrating through the housing 36. Thus, after the physician locates the housing 36, the physician may fully insert the syringe 26 needle into the housing 36 without great concern that the syringe 26 will pass through the housing 36. In addition, the access port 14 also provides the benefit that the outer layers of the housing 36 extend entirely around the fluid reservoir 24. The outer layers of the housing 36 provide a large penetrable surface for the physician to contact with the syringe 26. In the embodiment shown in FIG. 2, the entirety of the anterior side 40 of the housing 36 is needle penetrable. In addition, approximately 60% of the entire outer surface of the housing 36 is needle penetrable. The large penetrable surface area allows the physician to insert a needle into the access port 14 from a variety of different angles and make contact with the fluid reservoir 24. A greater penetrable surface area improves accessibility of the access port 14 for percutaneous needle insertion, which decreases the likelihood of multiple attempts at needle insertion and the accompanying pain and discomfort to patients. The accessible needle surface area of the access port 14 is preferably at least 0.45 square inches. It is contemplated this surface area may be increased or decreased to produce varied desired results.

The access port 14 provides other benefits, including the pliant and resilient composition of the housing 36. The elastomeric materials preferably composing the interior layer 46, and the exterior layer 50, are pliable and may deform when a force is exerted against the housing 36. The housing 36 is preferably firm enough to palpate when pressurized with fluid, yet is not a completely rigid structure. In addition, the silicone gel layer 48 provides a deformable cushion layer between the interior layer 46 and the exterior layer 50. The housing 36 therefore is not a stiff, or hard, device implanted within in the patient's body. The pliable nature of the housing 36 may increase total comfort of the device for the patient, as a rigid port within the patient's body may irritate local tissue surrounding the port 14. The pliable housing 36 may also reduce the chance of injury to the patient when forces are exerted on the housing 36, as the housing 36 will absorb a measure of the force exerted.

Another benefit of the embodiment shown in FIG. 2 includes the ellipsoid shape of the housing 36. The ellipsoid shape produces a large needle penetrable surface area for the physician to contact with a syringe 26 needle. The syringe 26 may not only be inserted from directly above the access port 14, but may also enter the access port 14 through a variety of incident angles. The variety of insertion points may be beneficial if the access port 14 twists or moves relative to the desired orientation within the body. In obesity treatments, movement of the access port 14 is a common concern, as the patient will hopefully undergo rapid weight loss, which may vary the position of the access port 14 relative to the surface of the patient's skin 30. A variety of entry points, positioned along both the anterior side 40 and the posterior side 42 of the housing 36 increases the total number of successful entry points for the access port 14.

The ellipsoid shape also provides the benefit that the housing 36 forms a bead-like object that can be easily identified by the physician through tactile means. A physician can run his or her hand along the surface of the patient's skin 30, and may detect the midline 44 of the access port 14 connecting the anterior side 40 and the posterior side 42 of the housing 36. The ellipsoid shape thus provides a unique, identifiable, structural presence that is more easily detectable within the patient's body than merely a half-dome or half-shell embodiment. The ellipsoid shape is thus beneficial when the housing 36 may be positioned within a thick fat layer 34 surrounding the access port 14. When used for obesity treatments, the three-dimensional ellipsoid shape enhances the possibility of detection for patients with large fat deposits.

In addition, the ellipsoid shape also forms a smooth contour shape beneath the patient's skin, which may increase the cosmetic, or aesthetic, effect of the housing 36. If the outline, or contour, of the housing 36 is visible through the surface of the patient's skin, the smooth contour produced by the ellipsoid shape may be more visually appealing than the outline produced by a rigid, angled, access port.

The use of a mesh anchor member 32 also provides benefits, including an enhanced degree of biocompatibility between the access port 14 and the patient's body. An expansive mesh provides a broad area for attaching the access port 14 to the patient's body through sutures, surgical tacks, or the like. Moreover, during post-operation healing, growing scar tissue becomes intertwined with the mesh, which enhances the security of the housing 36 to subcutaneous tissue. The more stable or secure the attachment, the less likely that the access port 14 may flip, invert, or dislodge, which lowers the difficulty of accessing the penetrable surface of the access port 14 for fluid injection or withdrawal. A stable attachment spares patients from pain associated with multiple attempts at injection, and reduces the need for reoperation to adjust the access port 14 position.

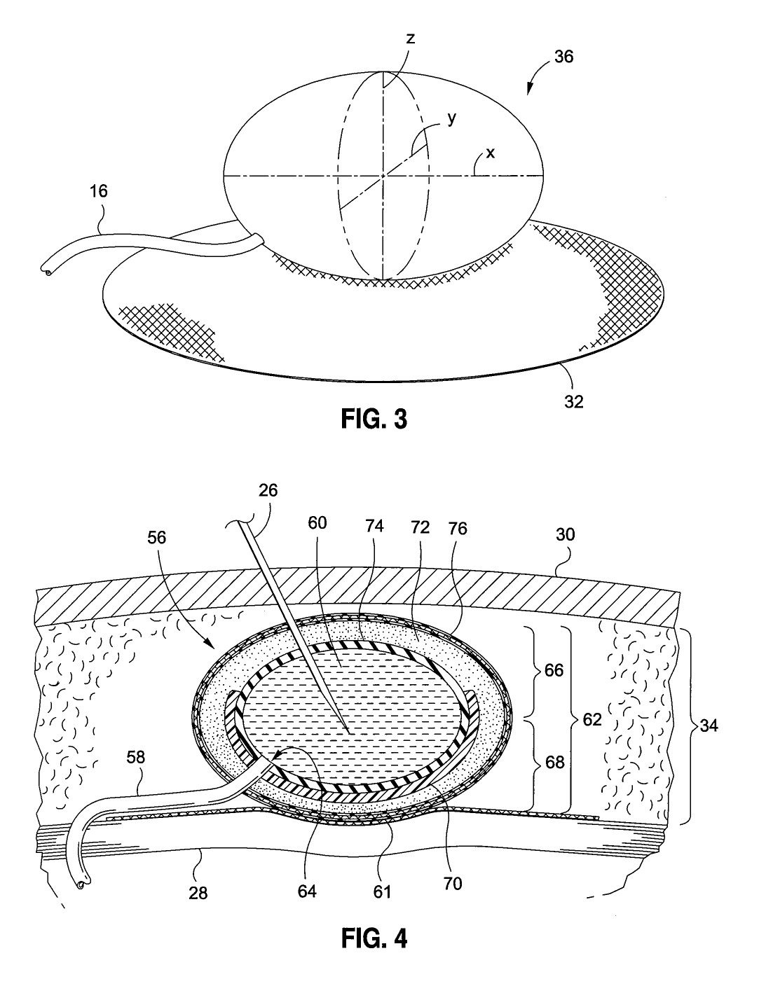

Referring to FIG. 3, the ellipsoidal shape of the housing 36 contemplates, but is not limited to shapes including a sphere and spheroid. Descriptions of ellipsoids typically involve reference to the x, y, and z axes of these structures. FIG. 3 illustrates a diagrammatic three-dimensional view of an embodiment of the housing 36 which shows the x, y, and z axes of the substantially ellipsoid-shaped housing 36. If the ellipsoid housing 36 were configured as a sphere, the housing 36 would have x, y, and z axes of equal length. If the ellipsoid housing 36 were configured as an oblate spheroid, the x and y axes would have the same length, the length being greater than the length of the z-axis. In one embodiment, each portion of the outer surface of the housing 36 has a curved local gradient, meaning each portion of the outer surface is curved. In one embodiment, only a portion of the outer surface of the housing 36 has a curved local gradient, but the housing 36 otherwise forms a substantially ellipsoid shape. In one embodiment, the anterior side 40 and the posterior side 42 (as defined in FIG. 2) of the housing 36 each have a domed, or half dome shape. In this embodiment, from the perspective of the fluid reservoir, the anterior side 40 and the posterior side 42 each have a convex shape. The housing 36 is not limited to an ellipsoid shape, but may have various non-ellipsoid shapes, for example, a half-dome, or half-shell shape, as desired.

As used in FIG. 3, the z-axis of the housing 36 refers to the height of the housing 36, or the dimension extending perpendicular to the surface of the muscle wall 28, as shown in FIG. 2. The x-axis and y-axis are orthogonal axes extending perpendicular to the z-axis, and extend substantially parallel to the surface of the patient's skin 30, as shown in FIG. 2. A plane formed by the x-axis and y-axis also extends substantially parallel to the surface of the patient's skin 30. The x-axis and y-axis define a width of the housing 36. In one embodiment, the width of the housing 36 along the x-axis and the y-axis is approximately equal, and may range between about 0.75 to about 1.75 inches, inclusive. In this embodiment, the height of the housing along the z-axis may range between about 0.60 and about 1.00 inches, inclusive. The defined widths and height of the housing 36 include the outer extent of the material layers comprising the housing 36. In one embodiment, the width of the housing 36 along the x-axis and the y-axis is equal, and is about 1.25 inches. In this embodiment, the length of the z-axis is about 0.80 inches. In one embodiment, the housing 36 may be shaped such that the width along the x-axis and the y-axis is unequal, and the width along the x-axis is greater than along the z-axis or the y-axis, as shown in FIG. 3. In other embodiments, the length along the axes may be varied as desired, to produce various sizes and shapes of the housing 36. The housing 36 may include any configuration of ellipsoid shapes, including sphere, or spheroid, or any other desired equivalent shapes that produce smooth curved contours of the housing 36 within the patient's body, or are capable as forming an outer surface for the housing 36.

FIG. 3 also illustrates the shape of the anchor member 32 in more detail than shown in FIG. 2. The anchor member 32 may extend out from the base of the housing 36 with a diameter being larger than that of the housing 36. The anchor member 32 may comprise a substantially flat sheet of mesh, that may have a disk-like, or skirt-like shape. The anchor member 32 may also be shaped or contoured to match the shape of the desired fixing point within the patient's body.

FIG. 4 illustrates a layer of needle resistant material 70 extending entirely around the portion of the fluid reservoir 60 on the posterior side 68 of the housing 62, and partially around the portion of the fluid reservoir 60 on the anterior side 66 of the housing 62. The needle resistant material 70 extends adjacent to the interior layer 74 of the housing 62, and between the interior layer 74 and the exterior layer 76. The needle resistant material 70 may be positioned within the gel layer 72 of the housing 62. A portion of the needle resistant material 70 includes a hole or passage, to allow the conduit 64 and the connecting tube 58 to pass therethrough. In the embodiment shown in FIG. 4, the total needle penetrable region of the housing 62 comprises approximately 40% of the total surface area of the housing 62. An increased size of needle resistant material 70 decreases the possibility that a needle passes through the housing 62, but also decreases the total needle penetrable surface area of the access port 56, and the housing 62.

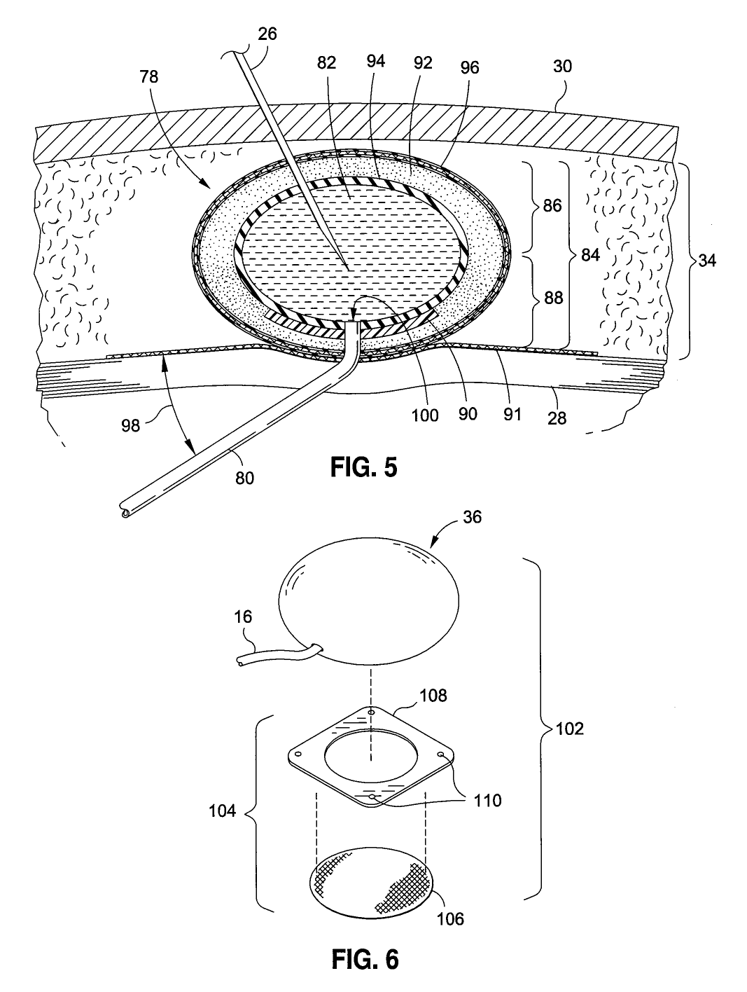

FIG. 5 illustrates a conduit 100 positioned at the lowest point of the fluid reservoir 82, or equivalently referred to as the lowest height of the fluid reservoir 82, or equivalently referred to as a point of lowest local surface curvature of the fluid reservoir 82, or otherwise referred to as the center or apex of the posterior side 88 of the housing 84. The conduit 100 is positioned at the center of the posterior side 88 of the housing 84 to allow a connecting tube 80 to pass through the center of the posterior side 88 of the housing 84. The connecting tube 80 extends through the layer of a needle resistant material 90 and through the anchor member 91. The needle resistant material 90 and the anchor member 91 are configured similarly to the embodiments shown in FIG. 2, aside from having holes, or passages, to accommodate passage of the connecting tube 80. The conduit 100 may also be positioned at any other equivalent position along the housing 84 that allows the tube 80 to pass through the needle resistant layer 90 and/or the anchor member 91.

The connecting tube 80 shown in FIG. 5 extends at an angle 98 from a plane extending parallel to the center of the posterior side 88 of the housing 84, to which the connecting tube 80 is attached. In the embodiment shown in FIG. 5, the plane is equivalently represented by the planar anchor member 91 extending from the housing 84. The connecting tube 80 extends in a posterior direction away from the housing 84, and away from the surface of the patient's skin 30. The angle 98 defined by the connecting tube 80 may be at an acute angle 98 between about 20 and about 50 degrees, inclusive, preferably at approximately 30 degrees. A benefit of extending the connecting tube 80 at an angle from the housing 84 is to lessen the likelihood that the connecting tube 80 causes damage to surrounding tissue, such as tissue and viscera in the abdominal cavity below the access port 78, after the access port 78 has been implanted subcutaneously. The connecting tube 80 extends at the angle 98 away from the housing 84, to reduce organ erosion and/or damage caused by the presence of the tube 80. A shallow angle of approximately 30 degrees may reduce the potential for organ erosion or damage caused by the tube 80. In addition, the connecting tube 80 extends below the skirt of the anchor member 91, which may serve to protect, shield, or cover, the tube 80 from errantly placed needles that do not contact the housing 84. The anchor member 91 may serve as a shield for the tube 80. The access port 78 shown in FIG. 5 includes an exterior layer 96, an intermediate gel layer 92 and an interior layer 94, similar to the embodiment shown in FIG. 2.

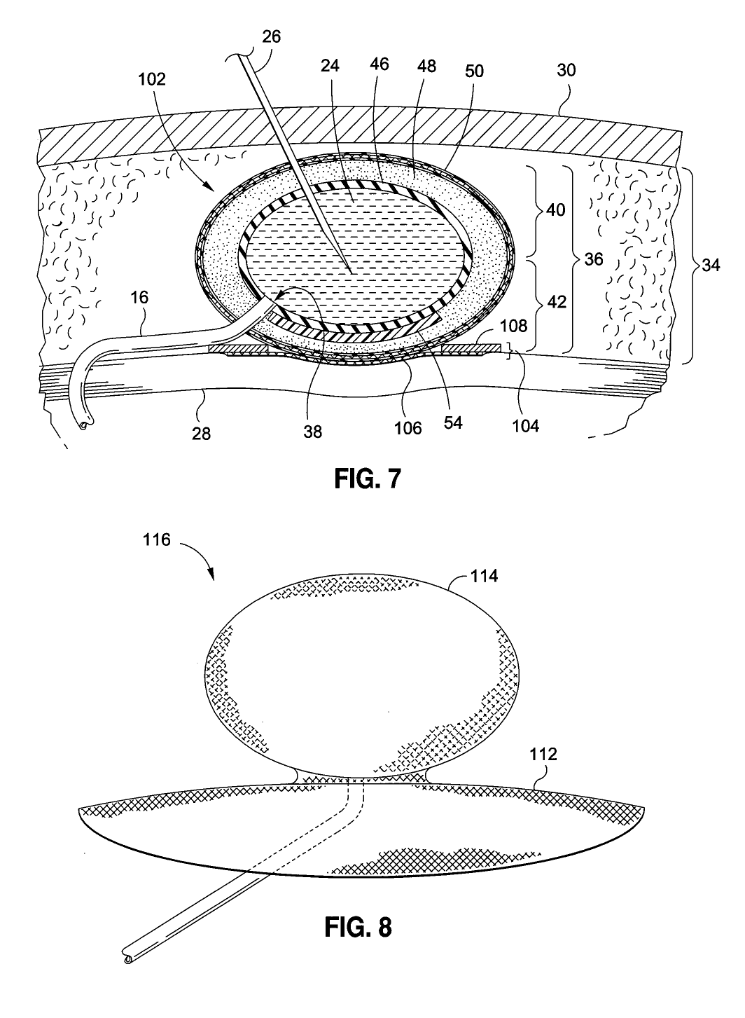

FIG. 6 illustrates an access port 102 with an anchor member 104 comprising a mesh member, or piece of mesh 106 coupled to a ring 108. The mesh 106 may be made from a similar mesh material as the mesh anchor member 32 described in relation to FIG. 2. The mesh 106 may have a circular, square, or other symmetric shape. The mesh 106 may be fused to the ring 108, for example, by ultrasonic welding. The ring 108 may have a rectangular or circular shape, with a substantially circular hole through the center of the ring 108. The ring 108 may be made of a hard, needle resistant material such as a plastic, or the like. The ring 108 may have apertures 110 on the corners or periphery of the ring 108. The apertures 110 may be configured for coupling the ring 108 to the desired portion of the patient's body. The apertures 110 may be shaped to receive sutures or other means to anchor the anchor member 104 to the desired portion of the patient's body. The mesh 106 may be circumscribed within the substantially circular hole within the ring 108, or an outer portion of the mesh 106 may overlap part of the ring 108 and attach to the ring 108, as shown in FIG. 7.

FIG. 7 illustrates a cross-section view of the anchor member 104 comprising the mesh 106 and the ring 108 shown in FIG. 6. The portion of the mesh 106 exposed through the circular hole of the ring 108 is bonded to the posterior side 42 of the housing 36. The ring 108 is not bonded directly to the housing 36, but rather connects to the patient's body, for example, through the apertures 110 shown in FIG. 6. In one embodiment, the ring 108 may also be directly bonded to the housing 36. In one embodiment, the mesh 106 may be attached directly to the patient's body, with or without the use of the ring 108. In this embodiment, the mesh 106 may attach to the patient's body through sutures, surgical tacks, or the like.

A benefit of the ring 108 and the mesh 106 combination is the ring 108 may protect the patient's body from further penetration by the syringe 26 needle if the needle misses the housing 36 or passes through a portion of the posterior side 42 of the housing 36 that is not protected with the layer of the needle resistant material 54. The ring 108 may protect the portion of the patient's body to which the housing 36 is attached (e.g., the muscle wall 28) from needle penetration in case of an errant needle stick by the physician. In addition, the ring 108 may be used in combination with the position of the conduit 100 and connecting the tube 80 shown in FIG. 5, to allow the ring 108 and the mesh 106 to provide additional protection for the tube 80 from errant needle sticks (e.g., the tube 80 will pass beneath the mesh 106, the ring 108, and will pass through the needle resistant layer 54).

FIG. 8 illustrates a mesh anchor member 112 being continuous, or contiguous, with the mesh layer 114 of the housing 116. In this embodiment, the mesh used in the anchor member 112 and the housing 116 comprises a single piece of mesh used to serve as an anchor member 112, and to provide structure for the housing 116. Accordingly, the material used to form the mesh anchor member 112 is the same as the material comprising the mesh layer 114 of the housing 116. The continuous material of the mesh anchor member 112 may extend over the entire housing 116, or may extend over only a portion of the housing 116. If the material of the mesh anchor member 112 only extends over a portion of the housing 116, a different mesh material may be used to cover the remainder of the housing 116, or may be entirely excluded. The continuous mesh material connecting the mesh anchor member 112 to the housing 116 increases the total strength and durability of the access port.

Exemplary Case Study of Gastric Band Treatment

A 41-year-old patient contemplates surgical means for the reduction of obesity. The patient with a high body mass index, for example 44 kg/m², may be classified as morbidly obese. The patient's medical history reveals no known comorbidities other than slightly elevated blood pressure. The patient's doctor considers the patient generally a good candidate for laparoscopic adjustable gastric banding.

During laparoscopic surgery, an inflatable gastric band, for example, a gastric band contemplated by the present invention, is inserted into the patient via small incisions in the abdomen, usually about 0.5-1.5 centimeters in length, using the pars flaccida technique. The inflatable gastric band is placed around the top portion of the patient's stomach, below the esophagogastric junction, creating a small pouch above the band that limits or reduces food consumption. The tubing extending from the gastric band is brought outside the abdomen. The access port, for example, an access port contemplated by the present invention, is securely connected to the gastric band. The conduit on the posterior side of the access port is joined to a stainless steel tubing connector, which is in turn connected to tubing extending from the gastric band so that the fluid introduced into the access port can be delivered through such tubing to inflate the gastric band. The access port is then placed on the rectus muscle or in another accessible subcutaneous site. The access port is then fixed in place by suturing or surgical tacking of an anchor member of the access port onto the left upper abdominal wall rectus fascia. A self-sealing, needle penetrable anterior side of the access port faces the skin, through which the syringe needle will be inserted during follow-up recalibrations of the gastric band.

In a follow-up appointment with the physician six weeks after the laparoscopic surgery, the physician locates the access port through palpation. The physician inserts a syringe needle through the skin covering the access port, and through the penetrable, self-sealing surface of the anterior side of the access port, to inject saline into a fluid reservoir in the access port. Saline fluid flows through the conduit, and the tubing connecting the access port to the gastric band. The fluid inflates and tightens the gastric band. The physician checks the appropriateness of the adjustment by having the patient drink water. If the patient is unable to swallow, the doctor removes saline fluid from the access port.

Alternative or Supplemental Design and Construction of Access Port Housing, and Methods of Manufacture

In one embodiment of the invention, an alternative or supplemental method of making the housing is provided wherein the method generally comprises the steps of providing a plurality of mesh segments, positioning the plurality of segments on a curved molding surface, applying a fluid elastomeric material to the molding surface with the segments positioned thereon, and allowing the elastomeric material to set to form a flexible shell having an open end, the shell including the fabric segments embedded within the set elastomer, and the shell being useful as the housing of the access port. The step of positioning may include substantially entirely covering the molding surface with the mesh segments, for example, in a manner such that the mesh segments overlap one another. The method further comprises the step of sealing the open end of the elastomeric shell, for example, by providing a puncture resistant material and sealing the puncture resistant material to the open end of the elastomeric shell.

In one embodiment, the mesh segments comprise a non-stretchable mesh fabric, for example, a substantially non-expanding polyester fabric mesh. In another embodiment, the mesh segments comprise a stretchable mesh fabric.

The method may further comprise the step of applying a tacky material to the curved molding surface prior to the step of positioning the mesh. The tacky material may be a fluid elastomeric material, for example, a silicone dispersion.

In another embodiment, the method comprises pre-shaping, for example, thermoforming, a mesh element, from a two-dimensional sheet into a three dimensional “sock” having the general shape of the molding surface. The method includes positioning the pre-shaped mesh element onto the molding surface, applying a fluid elastomeric material to the molding surface with the pre-formed mesh positioned thereon, and allowing the elastomeric material to set to form a flexible shell having an open end, the shell including the preformed mesh embedded within the set elastomer, and the shell being useful as a component of the housing of the access port.

The elastomeric material may be a silicone elastomer such as a dimethyl silicone elastomer, for example, a substantially homogeneous dimethyl-diphenyl silicone elastomer. One composition that may be useful in the present invention is described in Schuessler, et al., U.S. application Ser. No. 12/179,340, filed Jul. 24, 2008, the disclosure of which is incorporated herein in its entirety by this specific reference. The elastomeric material may comprise a room temperature vulcanizing (RTV) or a high temperature vulcanizing (HTV) silicone from about 0.1-95 wt %, for example, about 1-40 wt %, for example, about 30 wt %. In an exemplary embodiment, the silicone-based fluid material is a high temperature vulcanizing (HTV) platinum-cured silicone dispersion in xylene.

The mesh layer may comprise a mesh or fabric, for example, a synthetic polymer mesh or fabric, for example, a mesh or fabric made from poly(ethylene terephthalate) (PET), polypropylene (PP), polyurethane (PU), polyamide (Nylon), polyethylene (PE), any other suitable material, or combinations thereof.

In one embodiment, the exterior layer is made by dipping two or more layers of silicone-based elastomer over a conventional mandrel, followed by placement of a pre-fabricated 2 or 4-way stretchable “sock” of the mesh layer, followed by two or more dips of the silicone-based elastomer. The reinforcing “sock” is able to take the shape of the mandrel and the fabric is trapped on both sides between the elastomer layers. In this embodiment, the stretchable pre-shaped “sock” (which may form the reinforcing mesh layer 52 shown in FIG. 2A) can be relatively easily mounted on the mandrel because of its flexibility and elasticity, making it easier to manufacture a reinforced shell with the intended shape and dimensions of the mandrel. The entire assembly forming the exterior layer is heated in an oven at a temperature and time suitable to cure the silicone.

In one embodiment of the invention, the mesh layer is provided by forming a “sock” by using a cinch. Alternatively, the mesh layer is thermoformed into “sock” by placing a single sheet of suitable material, for example, a non-stretchable mesh, over a mandrel, and gathering the mesh material. The gathered mesh material is shaped, for example, thermoformed, to take on the 3-D shape of the mandrel.

Post-curing, the reinforced shell is removed from the mandrel, and another elastomeric shell (which forms the interior layer 46 shown in FIG. 2A) is placed inside the first shell (which forms the exterior layer 50 shown in FIG. 2A). The inner layer may be a typical unreinforced elastomeric shell, or alternatively may be made similarly to that described above with respect to the exterior layer. The interior layer may have the same or smaller size relative to the exterior layer. The dual-shell assembly is mounted back on a mandrel. The size of the mandrel can be the same as the one used for the inner shell fabrication or slightly larger. The latter results in a laterally stressed inner shell with potentially enhanced sealing properties.

In one embodiment, at least one of the interior layer and the exterior layer comprises an elastomeric material comprising a substantially homogenous layer of a silicone elastomer comprising a polysiloxane backbone and having a minimum mole percent of at least 10% of a substituted or pendant chemical group that sterically retards permeation of the silicone gel through the layer. More specifically, in this embodiment, the silicone elastomer is a polydimethyl siloxane and the pendant chemical group is one of a phenyl group, for example, a diphenyl group or a methyl-phenyl group, a trifluoropropyl group, and mixtures thereof. Such materials are described in detail in Schuessler, et al., U.S. application Ser. No. 12/179,340, filed on Jul. 24, 2008. This material may make up one or more layers of the interior layer or exterior layer.

After the interior layer and exterior layer are bonded together, a cavity formed therebetween is then filled with a material, for example, a flowable material, for example, a silicone gel. This may be accomplished using any suitable means known to those of skill in the art. In one embodiment, the gel is introduced through a plug on the exterior layer. The silicone gel between the exterior and interior layer forms the intermediate layer. After filling, the assembly made up of the interior layer, the exterior layer and the intermediate layer gel layer (similar to the gel layer 48 shown in FIG. 2A), is cured, for example, by exposing the assembly to heat in an oven for a suitable length of time. The mandrel that defines the desired shape of the housing can be round or oval, with a lower or upper pole for optimal projection. Before sealing the housing with a patch, a needle guard element, such as that described and shown elsewhere herein, may be inserted and bonded to the interior layer and/or the exterior layer.

Other compositions of the shell of the housing may include a dual lumen with pre-fabricated PET reinforced-gel sheeting. Incorporation of a reinforced gel sheeting of defined thickness (demonstrated self-sealing properties) bonded to the inside of a single standard textured shell. The key for the bonding of the gel sheeting to the inside of the shell is to coat and cure both sides of the gel sheeting with dispersion in order that employing an RTV adhesive, the coated gel will bond to smooth inner surface of a vulcanized textured shell.

Other compositions of the shell of the housing may include a molded housing with an elastomeric anterior self sealing surface. This concept utilizes a mold created to the shape of the housing. Elastomers placed into these molds in a combination of durometers and wall thickness provide surfaces that are self sealing by nature. The durometer ranges from about 30A to about 80A with a thickness greater than 0.080 inches for a self sealing surface. The molding can be done using standard techniques such as casting, liquid silicone rubber molding, thermoforming, etc.

Other compositions of the shell of the housing may include a shell embedded with micro-particles of swelling agents. Embedded into the silicone shell may be hydrogel particles that swell when in contact with liquid fillers (e.g., saline). Swelling applies tension/compressive forces onto the shell. The swelling agent may be PEG or PAA, PVA, PHEMA, ethyl cellulose or salts. See, for example, Schuessler, U.S. application Ser. No. 12/543,795, filed on Aug. 19, 2009, the disclosure of which is incorporated herein in its entirety by this specific reference. The mechanism of self sealing is facilitated by the swelling of the microparticles due to saline absorption from inside the implant.

Other compositions of the shell of the housing may include silicones of appropriate durometer used in the construction of self-sealing layers. A low durometer tacky silicone under tension is trapped between an interior and exterior shell layers. Or, alternating low and high durometer materials are cured under tension when forming the shell of the housing, in the relaxed state, the middle layers are under compression. As a needle is introduced and withdrawn this low durometer layer collapses around the void left by the needle and plugs the hole preventing leakage of filler liquid.

Other compositions of the shell of the housing may include a dense, flexible, closed cell foam attached to inner surface of shell. A combination of a dual shell construction encapsulating densely packed closed cell foam prevents the saline from escaping through a straight liquid channel. The compressive forces exerted by the densely packed cell layers of foam create a tortuous path for the liquid that tries to extrude to the exterior.

Other compositions of the shell of the housing may include a grafted or physical coating of hydrogel on the inside of the shell. To facilitate the self sealing effect, the implants are coated on the internal surface with a hydrogel. The hydrogel can be a polyacrylic acid based, polyvinyl alcohol based, polyhydroxyethyl methacrylate based or similar, or a combination of one or more hydrogels. The main requirements are a sufficiently high elongation at break and swell ratio.

Other compositions of the shell of the housing may include microparticles of aqueous hydrogel as filler. In this concept hydrogel particles would be placed inside the single lumen housing and these swell when the housing is filled with saline. Since the water is trapped in a hydrogel matrix, there is no availability of unbound water for leakage, after removal of the needle from the implant. This hydrogel can be a polyacrylic acid based, polyvinyl alcohol based, polyhydroxyethyl methacrylate based or similar, or a combination of one or more hydrogels. The main requirements are a sufficiently high elongation at break and swell ratio.

Other compositions of the needle resistant material may include thermoplastic films. Thin films (0.25-1 mm) of PC, PE, PP, PET, PS, Nylon and PTFE were found to resist 21 g needle puncture. These sheets can be manufactured with grooves in their design to allow for movement during insertion. They are attached using adhesives to the posterior shell of the device or alternatively are encapsulated in silicone which is vulcanized to the posterior shell layer for anchorage.

Other compositions of the needle resistant material include a flexible but relatively rigid self sealing elastomer. Thermoplastic polyurethanes (TPU's) and silicones of certain durometers have a good combination of flexibility and hardness, and are not as stiff as the previously mentioned thermoplastics. They are self-sealing in specific ranges of thickness (>1 mm) and durometer (>50A) which makes them an ideal candidate for a flexible self sealing needle guard. They may allow some minimal needle tip penetration, but can provide tactile feedback required to detect the needle guard and possess the flexibility to allow a smaller incision for insertion of the housing.

Other compositions of the needle resistant material include puncture resistant fabrics. The needle resistant layer can be fabricated from a pliable fabric that is puncture, cut and tear resistant. Such fabrics are seeing increasing use in the manufacture of gloves, hunting/military and medical apparel. Such a fabric is attached to the posterior of the device using adhesives or silicone encapsulation as a needle stopping component.

Other compositions of the needle resistant material include a combination of rigid and self sealing materials. The needle guard can be a combination of a self sealing elastomer with a rigid puncture resistant material such as the thermoplastics and the puncture resistant fabrics mentioned earlier. The thickness of the two layers can be controlled to develop a needle resistant layer that provides resistance to puncture, is self sealing and flexible enough such that the housing may be easily inserted through a small incision.

Other compositions of the needle resistant material include foldable flanges in a needle stop. The concept is overlapping flexible flanges made from thermoplastics, for example, polypropylene. The flanges allow a partially foldable or manipulable (for insertion and removal) needle resistant layer, and the polypropylene is impenetrable.

The various access port embodiments described throughout this application are not limited to use in a gastric band system, or for the treatment of obesity. It is contemplated the access port may be used in various other medical applications, including other medical implantable devices, including skin expanders, or drug delivery systems.

Unless otherwise indicated, all numbers expressing quantities of ingredients, components, forces, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, certain references have been made to patents and printed publications throughout this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. An implantable access port for use with a gastric band for the treatment of obesity, the access port comprising:
a substantially ellipsoid-shaped housing defining an internal fluid reservoir and a conduit for the movement of fluid into and out of the internal fluid reservoir, at least a portion of the housing including a mesh layer; and
an anchor member coupled with the housing for attachment of the access port to body tissue.

2. The access port of claim 1, wherein the housing is substantially oblate spheroid-shaped.

3. The access port of claim 1, wherein the housing is substantially sphere-shaped.

4. The access port of claim 1, wherein the internal fluid reservoir is substantially ellipsoid-shaped.

5. The access port of claim 1, wherein the housing is defined by three orthogonal axes being an x-axis, a y-axis, and a z-axis, the housing having an outer width along the x-axis of between 0.75 to 1.75 inches, inclusive, the housing having an outer width along the y-axis of between 0.75 to 1.75 inches, inclusive, and the housing having an outer height along the z-axis of between 0.60 to 1.00 inches, inclusive.

6. The access port of claim 1, wherein the housing is pliant.

7. The access port of claim 1, wherein the housing has an anterior side and a posterior side, a portion of the anterior side being needle penetrable and self-sealing, a portion of the posterior side being resistant to needle penetration.

8. The access port of claim 7, wherein the anterior side of the housing has a dome-like shape and the posterior side of the housing has a dome-like shape.

9. The access port of claim 7, wherein the portion of the anterior side of the housing is made from an elastomeric material.

10. The access port of claim 7, wherein the portion of the posterior side of the housing is made from a fabric.

11. The access port of claim 7, wherein the anterior side of the housing includes the mesh layer, and the posterior side of the housing includes the mesh layer.

12. The access port of claim 11, wherein the mesh layer of the anterior side of the housing is contiguous with the mesh layer of the posterior side of the housing.

13. The access port of claim 1, wherein the housing includes an exterior layer of elastomeric material, an intermediate layer of silicone gel, and an interior layer of elastomeric material, the mesh layer being embedded in the exterior layer of elastomeric material.

14. The access port of claim 1, wherein the anchor member comprises a mesh member.

15. The access port of claim 14, wherein the mesh layer is contiguous with the mesh member.

16. The access port of claim 1, wherein the anchor member comprises a substantially circular piece of mesh.

17. The access port of claim 1, wherein the anchor member comprises a mesh member coupled to a ring.

18. The access port of claim 1, further comprising a connecting tube extending from the conduit.

19. The access port of claim 18, wherein the connecting tube extends away from the housing at an angle that is between 20 to 50 degrees, inclusive.

20. A gastric banding system for the treatment of obesity comprising:
an adjustable gastric band including an inflatable portion;
a connecting tube extending from the inflatable portion; and
an access port, including:
a substantially ellipsoid-shaped housing defining an internal fluid reservoir and a conduit for the movement of fluid into and out of the internal fluid reservoir and being coupled with the connecting tube, at least a portion of the housing including a mesh layer; and
an anchor member coupled with the housing for attachment of the access port to body tissue.
